(19) United States
(12) Patent Application Publication
(10) Pub. No.: US 2026/0297454 A1
(43) Pub. Date: Oct. 1, 2026

(54) PROCESS FOR DEGUMMING FEEDSTOCK COMPRISING BRASSICA CARINATA OIL

(71) Applicant: DSM IP ASSETS B.V., Maastricht (NL)

(72) Inventors: Ying ZHA, Echt (NL); Weixi QIU, Echt (NL); Ling HUA, Søborg (DK)

(21) Appl. No.: 19/475,945

(22) PCT Filed: Apr. 19, 2024
(86) PCT No.: PCT/EP2024/060799

§ 371 Date: Oct. 16, 2025

(30) Foreign Application Priority Data
Apr. 20, 2023 (EP) 23169007.4

(51) Int. Cl. C11B 3/00; C11B 3/10; C12N 9/20

(52) U.S. Cl. CPC C11B 3/003; C11B 3/001; C11B 3/10; C12N 9/20; C12Y 301/01032

(57) ABSTRACT

The present invention relates to a process degumming and refining crude Brassica carinata oils and feedstocks comprising such crude oils. The process comprises a step of incubating the feedstocks with a fungal phospholipase A1 having at least 50%, more preferably at least 75%, sequence identity with SEQ ID NO: 1. The process can further comprise incubating the feedstocks with a fungal phospholipase A1 in the presence of an acid, separating the phosphorous-containing components from the processed feedstock, e.g. by centrifugation, and/or an adsorption step using e.g. bleaching earth. The invention further relates to feedstocks comprising B. carinata oil or B. carinata oils that have been degummed using the process of the invention, as well as the use of a fungal phospholipase A1 for degumming and/or refining of crude B. carinata oils and feedstocks comprising such crude oils.

### FIELD OF THE INVENTION

The present invention relates to the fields of enzymology and vegetable oil processing. In particular the invention relates to the enzymatic degumming of Carinata oil, preferably using a process wherein crude Carinata oil is incubated with a fungal-derived phospholipase A1.

### BACKGROUND OF THE INVENTION

The global demand for vegetable-based oils continues to rise, while the availability of highly productive arable farmland is becoming progressively limited. To meet the requirements of the future, it will be essential to develop new and improved temperate oilseed cultivars adapted to less-than-optimum acreage.

Brassica carinata is a species that is well-adapted to growth in semi-arid regions and is highly drought tolerant. It is a crop that comes close to rapeseed, but is not used in food, therefore there is no ‘food vs fuel’ discussion. B. carinata grows faster than the ordinary rapeseed crop, and can be planted as ‘in between’ crops, so there is also no ‘forest vs. farmland’ discussion.

B. carinata is therefore being developed as a new crop platform dedicated to the production of bio-industrial oil feedstocks, most notably oils enriched in the very long-chain fatty acids (VLCFAs) erucic and nervonic. VLCFA-enriched B. carinata oils have applications in the manufacture of bio-jet fuels, biodiesel, enhanced oil recovery (EOR) surfactants, bioplastics and many other products.

For the production of such biodiesel or aviation biofuel from B. carinata oil, reduction of phosphorus, calcium and/or magnesium levels would be advantageous. As the oil is mainly used for hydrotreated vegetable oil (HVO) or Renewable Diesel HVO production, the oil that goes into HVO reactor has to be meet specifications on phosphorous and metal contents.

Traditionally such a reduction on phosphorus, calcium and/or magnesium levels is carried out chemically (see e.g.: https://lipidlibrary.aocs.org/edible-oil-processing/chemical-degumming). However, even though B. carinata oil comes close to rapeseed oil, in the conventional chemically refining process it behaves differently than rapeseed oil. Conventional chemical refining of B. carinata oil is unable to sufficiently reduce phosphorus and metal levels to HVO reactor specifications.

Enzymatic degumming of vegetable oils such as soybean, sunflower, canola, etc., using phospholipases (Cerminati et al., 2019, Appl. Microbiol. Biotechnol. 103:2571-2582); or using phospholipase A1 (WO 2019/215078); or using a combination of a lipase and a phospholipase (WO 2022/233897), has been described previously. The total content of phospholipids and their composition in crude oil differ according to the origin of the vegetable oil. As different phospholipids species have different chemical properties, phospholipid composition of the oil as well as the choice of enzyme greatly affect the efficiency of enzymatic oil degumming (see e.g. Steinke et al., 2022, BioEnergy Res. 15:1555-1567). No reports have issued about the efficiency and enzymes of choice for degumming of B. carinata oil.

US2020/0017775 relates to a process for producing hydrocarbons, where feedstock of biological origin is co-processed with non-renewable feedstock in a catalytic cracking process. It describes, amongst others, a process, comprising a step wherein feedstock of biological origin is degummed to produce degummed feedstock. In one embodiment it is mentioned that the feed-stock of biological origin can be selected from mixtures of plant oil(s), where the mixture may comprise 10%-100% of plant oil originating from Brassica carinata. In passing, enzymes are mentioned, but no specific phospholipase enzyme is taught. In the examples preference is given to chemical acid degumming at about 80° C. U.S. Pat. No. 11,046,905 describes another method for producing renewable fuels and renewable fuel components, where the method comprises processing of Brassica carinata seeds. Also here the examples illustrate acid degumming of the seed oil at about 80° C. The high temperature and generated waste streams are less desirable from an economic as well as environmental viewpoint.

There is therefore a need in the art for a process for refining B. carinata oil that sufficiently reduce phosphorus and metal levels to HVO reactor specifications. It is thus an object of the invention to provides means and methods for such refining process and to provide for oil obtained in such processes. In addition, it may be desirable that such a process is more economically and/or environmentally acceptable.

### SUMMARY OF THE INVENTION

The above object has been resolved by the process according to the invention.

Accordingly, in a first aspect the invention provides a process for reducing an amount of at least one of intact phospholipids and metals in a feedstock comprising oil from Brassica carinata, the process comprising the step of incubating the composition with a polypeptide having phospholipase A1 activity, wherein the polypeptide comprises an amino acid sequence having at least 50%, more preferably at least 75%, sequence identity to SEQ ID NO: 1. In one embodiment, the polypeptide reduces at least 85% of the amount of intact phospholipids originally present in a crude B. carinata oil when the polypeptide is incubated with the crude B. carinata oil in an amount of 2500 PLAU/kg oil in the presence of 300-500 ppm citric acid, at a temperature of 60° C. for 3 hours. In one embodiment, the polypeptide is an Aspergillus phospholipase A1, preferably an A. niger phospholipase A1, more preferably the polypeptide comprises the amino acid sequence of SEQ ID NO: 1.

In one embodiment of the process of the first aspect, the feedstock is incubated with the polypeptide having phospholipase A1 activity in the presence of an acid, wherein, preferably the acid is an acid selected from the group consisting citric acid, phosphoric acid, acetic acid, tartaric acid, succinic acid, and mixtures thereof. In one embodiment, the acid is present in an amount of 100-1000 ppm, preferably 200-800 ppm, more preferably 400-600 ppm, and most preferably 400-600 ppm citric acid.

In one embodiment of the process of the first aspect, the feedstock is incubated with the polypeptide having phospholipase A1 activity at a temperature in the range of 50-70° C., preferably at temperature in the range of 55-65° C.

In one embodiment of the process of the first aspect, the process comprises a further step of separating the phosphorous-containing components from the processed feedstock, preferably by centrifugation.

In one embodiment of the process of the first aspect, the process comprises a further step of combining a processed feedstock with an adsorption medium to generate a slurry and separating the adsorbed processed feedstock from the adsorption medium. In one embodiment, the adsorption medium is a bleaching earth.

In one embodiment of the process of the first aspect, the process is a process for degumming and/or refining the feedstock.

In one embodiment of the process of the first aspect, the feedstock comprises crude B. carinata oil, or wherein preferably the feedstock is crude B. carinata oil.

In a second aspect the invention provides a degummed feedstock comprising B. carinata oil, wherein the feedstock is characterized by at least one of: a) a phosphorus content of less than 10, 5, 2 or 1 ppm, preferably as determined by ³¹P NMR; and, b) a total metals content of less than 10, 5, 2 or 1 ppm, preferably as determined by an ICP spectroscopic method, and wherein preferably the degummed feedstock is degummed B. carinata oil.

In one embodiment, the degummed feedstock or the degummed B. carinata oil is characterized by at least one of: i) a content of calcium of less than 10, 5, 2 or 1 ppm, preferably as determined by an ICP spectroscopic method; ii) a content of iron of less than 2, 1, 0.5 or 0.2 ppm, preferably as determined by an ICP spectroscopic method; and, iii) a content of magnesium of less than 5, 2, 1, 0.5 or 0.2 ppm, preferably as determined by an ICP spectroscopic method.

In one embodiment, the degummed feedstock or the degummed B. carinata oil is obtainable in a process according to the first aspect.

In a third aspect the invention provides a use of a polypeptide having phospholipase A1 activity as defined above, for at least one of: a) degumming of; b) refining of; c) reducing the level of phosphorus in; and, d) reducing the level of metals in, a feedstock comprising oil from B. carinata, wherein preferably the feedstock comprises crude B. carinata oil, or wherein, more preferably the feedstock is crude B. carinata oil.

The invention can advantageously allow for a process that can be more economically and/or environmentally acceptable.

### BRIEF DESCRIPTION OF THE SEQUENCE LISTING

This application contains a Sequence Listing in computer readable form, which is incorporated herein by reference. An overview is provided below.

| SEQ ID NO: | Enzyme Name | Origin | Type |
|---|---|---|---|
| SEQ ID NO: 1 | phospholipase A1 | Aspergillus niger | protein |

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

For purposes of the present invention, the following terms are defined below.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g., about 10) preferably means that the value may be the given value (of 10) more or less 10% of the value.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g., Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (polynucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTP programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic | Ser (S), Thr (T), Asn (N), |
| Uncharged Residues | and Gln (Q) |
| Aliphatic | Gly (G), Ala (A), Val (V), |
| Uncharged Residues | Leu (L), and Ile (I) |
| Non-polar | Cys (C), Met (M), and Pro (P) |
| Uncharged Residues | |
| Aromatic Residues | Phe (F), Tyr (Y), and Trp (W) |

### Alternative Conservative Amino Acid Residue Substitution Classes.

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues.

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, |
| | T, V, W, and Y |
| Negatively charged residues | D and E |
| Polar residues | C, D, E, H, K, N, Q, R, |
| | S, and T |
| Positively charged residues | H, K, and R |
| Small residues | A, C, D, G, N, P, S, T, and V |
| Very small residues | A, G, and S |
| Residues involved | A, C, D, E, G, H, K, N, Q, |
| in turn formation | R, S, P and T |
| Flexible residues | Q, T, K, S, G, P, D, E, and R |

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly(A) stretch or the complement thereof (e.g., practically any double-stranded cDNA clone).

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed earlier herein.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which it is introduced but have been obtained from another cell or synthetically or recombinantly produced. Generally, though not necessarily, such nucleic acids encode proteins, i.e., exogenous proteins, that are not normally produced by the cell in which the DNA is transcribed or expressed. Similarly exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e., combinations where at least two of the combined sequences are foreign with respect to each other. The terms heterologous and exogenous specifically also apply to non-naturally occurring modified versions of otherwise endogenous nucleic acids or proteins.

The “specific activity” of an enzyme is herein understood to mean the amount of activity of a particular enzyme per amount of total host cell protein, usually expressed in units of enzyme activity per mg total host cell protein. In the context of the present invention, the specific activity of a particular enzyme may be increased or decreased as compared to the specific activity of that enzyme in an (otherwise identical) wild type host cell.

Filamentous fungi are herein defined as eukaryotic microorganisms that include all filamentous forms of the subdivision Eumycotina and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic.

A “mature polypeptide” is defined herein as a polypeptide in its final form and is obtained after translation of a mRNA into polypeptide and post-translational modifications of said polypeptide. Post-translational modifications include N-terminal processing, C-terminal truncation, glycosylation, phosphorylation and removal of leader sequences such as signal peptides, propeptides and/or prepropeptides by cleavage.

A phospholipid is also indicated as a glycerophospholipid. A phospholipid as used herein is an “intact” phospholipid and comprises a glycerol backbone comprising two fatty acids and a phosphoric acid. A phospholipid is also indicated as diacylglyceride comprising a phosphate group.

A lysophospholipid is a glycerol backbone comprising only one acyl (fatty acid) group, and a phosphate group. A lysophospholipid can be formed after removal of an acyl group from phospholipids by the action of a phospholipase A1, and/or a phospholipase A2.

The wording triacylglyceride oil and triglyceride oil are used interchangeably herein. A triglyceride is an ester derived from glycerol and three fatty acids. A triacylglyceride oil can be an edible oil and/or an oil used as a biodiesel.

Percentages and parts-per-million (ppm) given herein are in weight-per-weight unless otherwise indicated.

### DETAILED DESCRIPTION OF THE INVENTION

The present inventors have surprisingly found that whereas conventional chemical refining of Brassica carinata oil is unable to sufficiently reduce phosphorus and metal levels to HVO reactor specifications, a simple treatment of crude B. carinata oil with a fungal phospholipase A1 is much more efficient in reducing phosphorus and metal levels in the oil. A further adsorption step then suffices to bring the enzyme-treated oil up to HVO reactor specifications.

In a first aspect, therefore, the invention relates to a process for reducing an amount of at least one of intact phospholipids and metals in a feedstock comprising oil from B. carinata. The process preferably comprises the step of incubating the feedstock with a polypeptide having phospholipase A1 activity, wherein the polypeptide comprises an amino acid sequence having at least 50%, more preferably at least 75%, identity to SEQ ID NO: 1.

The processes described herein can accommodate a wide range of feedstocks comprising B. carinata oil. The B. carinata oil comprised in the feedstock can be classified as crude, degummed, and RBD (refined, bleached, and deodorized) grade, depending on level of pretreatment and residual phosphorus and metals content. In principle, feedstocks comprising any of these grades of B. carinata oil can be subjected to a process as described herein for further phosphorus and metals content. In one embodiment, a feedstock comprising B. carinata oil to be subjected to a process as described herein can be a mixture of B. carinata oil with other oils, e.g., other vegetable oils. In one embodiment, a feedstock comprising B. carinata oil to be subjected to a process as described herein comprises (in order of increasing preference) at least 10, 20, 30, 40, 50, 60, 70, 80, 90, 95 or 100% B. carinata oil, preferably at least 10, 20, 30, 40, 50, 60, 70, 80, 90, 95 or 100% crude B. carinata oil. Brassica carinata, commonly called Ethiopian mustard (known locally as gomenzer), is an amphidiploid formed through interspecific hybridization between the diploid parent species B. nigra L. and B. oleracea L. (Prakash and Hinata, 1980, “Taxonomy, cytogenetics and origin of crop Brassicas, a review”, Opera Botanica (Sweden). no. 55).

Crude vegetable oils such as crude B. carinata oil are obtained from pressing and/or solvent extraction methods and are a complex mixture of triacylglycerols, phospholipids, sterols, tocopherols, free fatty acids, trace metals, and other minor compounds. In commonly known processes, the seeds are first pressed leading to a pressed oil fraction. The press cake can be further treated with a solvent to yield an extracted oil fraction and the two fractions combined are known as crude oil for B. carinata. Crude B. carinata oil, also referred to as non-degummed oil, is herein thus understood to refer to a pressed and/or extracted and non-degummed B. carinata oil.

A polypeptide having phospholipase A1 activity as used herein is a phospholipase A1 according to enzyme classification E.C. 3.1.1.32. A phospholipase A1 is an enzyme that cleaves a phospholipid at the SN1 position forming a lysophospholipid and a fatty acid. A phospholipase A1 as used herein may also cleave a lysophospholipid at the SN1 position forming a glycerophosphate and a fatty acid. The wording “phospholipase A1” and a “polypeptide having phospholipase A1 activity” is used interchangeably herein. A polypeptide having phospholipase A1 activity as disclosed herein preferably does not have phospholipase A2 activity.

In one embodiment, the polypeptide having phospholipase A1 activity reduces at least 85, 86, 87, 88, 89, 90, 91, 92, 93, 94, or at least 95% % of the amount of intact phospholipids originally present in a crude B. carinata oil when the polypeptide is incubated with the crude B. carinata oil in an amount of 2500 PLAU/kg oil in the presence of 300-500 ppm citric acid, at a temperature of 60° C. for 3 hours, for example as tested and using assays as described in the Examples herein. “PLAU” refers to phospholipase A1 units as defined and determined in WO 2019/215078. Phosphorous components such as phospholipids, lysophospholipids and phosphate esters can be determined using ³¹P-NMR and/or HPLC, as are known in the art, for instance as disclosed in the Examples herein.

In one embodiment, the polypeptide having phospholipase A1 activity reduces at least 85, 86, 87, 88, 89, 90, 91, 92, 93, 94, or at least 95% % of the amount of metals, preferably at least one of calcium, magnesium and iron, originally present in a crude B. carinata oil when the polypeptide is incubated with the crude B. carinata oil in an amount of 0.6 mg active protein/kg oil in the presence of 300-500 ppm citric acid, at a temperature of 60° C. for 3 hours, for example as tested and using assays as described in the Examples herein. Metals content can be determined using Inductively Coupled Plasma (ICP) spectroscopic methods such as ICP-AES (atomic emission spectroscopy) and ICP-OES (optical emission spectroscopy), such as AOCS, as are known in the art, for instance as disclosed in the Examples herein.

Reducing the amount of metals is herein understood as a reduction of the total metals. Such metals can include, but are not limited to, As, Ca, Cr, Cu, Fe, K, Li, Mg, Mn, Na, Pb, Sr, Zn, or a combination of any two or more thereof. For example, in any embodiment herein, the total metals can include Ca, Fe, K, Mg, and Na, or can at least include Ca, Fe and Mg.

In one embodiment, the polypeptide having phospholipase A1 activity is a phospholipase A1 derived from a fungus, preferably from a filamentous fungus, more preferably from a filamentous fungus of the genus Aspergillus, most preferably from a filamentous fungus of the subgenus Circumdati, section Nigri. A suitable polypeptide having phospholipase A1 activity can thus be phospholipase A1 derived from a filamentous fungus of the genus Aspergillus selected from the group consisting of A. niger, A. tubingensis, A. foetidus, A. carbonarius, A. awamori, A. ficuum, A. saitoi, A. phoenicis, A. welwitschiae, A. piperis, A. vadensis, A. eucalypticola, A. luchuensis, A. neoniger, A. costaricaensis, A. brasiliensis, A. ibericus, A. sclerotiicarbonarius, A. sclerotioniger, A. ellipticus, A. homomorphus, A. heteromorphus, A. pseudoheteromorphus, A. japonicus, A. rambellii, A. lacticoffeatus, A. ochraceoroseus, A. aculeatus, and A. ellipticus.

In one embodiment, the polypeptide having phospholipase A1 activity is a polypeptide comprising an amino acid sequence having (in order of increasing preference) at least 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to SEQ ID NO: 1. In a preferred embodiment, the polypeptide having phospholipase A1 activity is a polypeptide comprising the amino acid sequence set forth in SEQ ID NO: 1. The amino acid sequence set forth in SEQ ID NO: 1 is the amino acid sequence of the mature phospholipase A1 from A. niger CBS 513.88, that comprises amino acids 30-298 of the pre-pro-phospholipase A1 amino acid sequence of Genbank accession no. XM_001393495.2. It is understood herein that the polypeptide having phospholipase A1 may lack a few amino acids from the N- and/or C-terminus as compared to a polypeptide having an above-defined percentage of sequence identity to SEQ ID NO: 1, due to exoproteolytic activity during the manufacture of the enzyme.

In one embodiment, the polypeptide having phospholipase A1 activity is a naturally occurring polypeptide. In one embodiment, the polypeptide having phospholipase A1 activity is a variant or modified polypeptide, having one or more amino acid substitutions, insertions and/or deletions as compared to a corresponding naturally occurring polypeptide.

Means and methods for producing a polypeptide having phospholipase A1 activity as described herein are generally known in the art and are e.g., described in WO 2019/215078.

Incubating a feedstock comprising B. carinata oil with a polypeptide having phospholipase A1 activity in a process as disclosed herein comprises converting phospholipids in the oil into lysophospholipids, and free fatty acids. Incubating the feedstock with a polypeptide having phospholipase A1 activity in a process as disclosed herein may further comprise converting phospholipids and/or lysophospholipids in the feedstock into lysophospholipids and/or glycerophosphates, and free fatty acids.

In one embodiment, incubating a feedstock comprising B. carinata oil with a polypeptide having phospholipase A1 activity in a process as disclosed herein is performed at a pH value of from 2 to 8, for instance from 3 to 7, for instance from 4 to 6.

In one embodiment, incubating a feedstock comprising B. carinata oil with a polypeptide having phospholipase A1 activity in a process as disclosed herein is performed in the presence of an acid. Accordingly, a process for reducing an amount of at least one of intact phospholipids and metals in a feedstock comprising B. carinata oil as disclosed herein comprises adding an acid such that the amount of acid in the feedstock is from 100 to 1000 ppm of acid, such as from 200 to 900 ppm of acid, for instance from 300 to 800 ppm of acid, for instance from 400 to 600 ppm of acid. A suitable acid used in a process as disclosed herein is an acid selected from the group consisting citric acid, phosphoric acid, acetic acid, tartaric acid, succinic acid, and mixtures thereof.

Usually, water is present in a process as disclosed herein. A process as disclosed herein may further comprise adding water to the oil, for instance an amount of water is added such that an amount of 0.5 to 5 wt %, such as an amount of 1 to 4 wt %, such as an amount of 2 to 3 wt % of water is present in the oil in a process as disclosed herein.

In one embodiment, incubating a feedstock comprising B. carinata oil with a polypeptide having phospholipase A1 activity in a process as disclosed herein, is performed at a temperature in the range of 40° C. to 75° C., preferably a temperature in the range of 50° C. to 70° C., more preferably as a temperature in the range of 55° C. to 65° C. and most preferably as a temperature in the range of 57° C. to 63° C.

A suitable period for incubating a feedstock comprising B. carinata oil with a polypeptide having phospholipase A1 activity in a process as disclosed herein, is from 0.5 to 10 hrs, for instance from 1 to 8 hrs, such as from 2 to 6 hrs, or from 2.5 to 4 hours.

In one embodiment of a process as disclosed herein, the feedstock comprising B. carinata oil is incubated with a polypeptide having phospholipase A1 activity as described herein, as sole lipolytic enzyme. Thus, when the feedstock comprising B. carinata oil is incubated with a polypeptide having phospholipase A1 activity as described herein, no further lipases and/or (lyso)phospholipases (A2, B, C or D) need be present.

In one embodiment, a process of incubating a feedstock comprising B. carinata oil with a polypeptide having phospholipase A1 activity as disclosed herein, comprises a further step of separating at least one of the phosphorous-containing components and metal-containing components, from the processed feedstock as obtained after incubation of the feedstock with the polypeptide. In one embodiment, the step of separating at least one of the phosphorous-containing components and metal-containing components, from the processed feedstock is performed by centrifugation. The centrifuging can include use of a disc-stack centrifuge, a decanter centrifuge, and/or a 3-phase centrifuge. Other methods, systems, and apparatus for separating and/or centrifuging the processed feedstock can be included, such as methods, systems, and apparatus such as settling tanks and are known to the skilled person.

In one embodiment, a process of incubating a feedstock comprising B. carinata oil with a polypeptide having phospholipase A1 activity as disclosed herein, further comprises an adsorption step. Thus in one embodiment, a process of incubating a feedstock comprising B. carinata oil with a polypeptide having phospholipase A1 activity as disclosed herein, comprises a step wherein the processed feedstock as obtained after incubation of the feedstock with the polypeptide, optionally further processed by separating at least one of the phosphorous-containing components and metal-containing components, is combined with an adsorption medium to generate a slurry, and wherein the adsorbed processed feedstock is subsequently separated from the adsorption medium.

Suitable adsorption media (also referred to herein as “sorbent media”) can include, but are not limited to, silica (e.g., silica hydrogels, silica hydrogel particles), diatomaceous earth, activated carbon, bleaching earths (also referred to as bleaching clays), perlite, cellulosic media, bauxite, silica aluminates, natural fibers, natural flakes, synthetic fibers, or a combination of any two or more thereof. In a preferred embodiment, the adsorption medium is a bleaching earth.

Means and methods for performing an adsorption step on oil feedstocks are described in more detail in e.g., U.S. Pat. No. 11,459,523.

In one embodiment, a process of incubating a feedstock comprising B. carinata oil with a polypeptide having phospholipase A1 activity, as disclosed herein, is a process for at least one of degumming and refining the feedstock. Crude vegetable oils such as B. carinata oil contain lecithins, phospholipids, and metals, which are generally called, because of their appearance, mucilaginous gums or simply “gum”. A process of elimination of these lecithins, phospholipids, and metals, i.e. gums is herein understood as “degumming”. It is further understood that a process of refining a vegetable oil, such as a feedstock comprising B. carinata oil, at least includes degumming of the vegetable oil and can further include one or more of adsorption, bleaching, deodorizing, water washing, FFA stripping, glycerolysis, and alkalinity reduction.

In one embodiment, a process of incubating a feedstock comprising B. carinata oil with a polypeptide having phospholipase A1 activity, as disclosed herein, can thus further comprise one or more of bleaching, deodorizing, water washing, FFA stripping, glycerolysis, and alkalinity reduction.

In one embodiment, a process of incubating a feedstock comprising B. carinata oil with a polypeptide having phospholipase A1 activity as disclosed herein, the feedstock comprises crude B. carinata oil, or wherein preferably the feedstock is crude B. carinata oil.

In a second aspect, there is provided a degummed feedstock comprising B. carinata oil. In one embodiment, the degummed feedstock is characterized by at least one of: a) a phosphorus content of less than 10, 5, 2 or 1 ppm, preferably as determined by ³¹P NMR; and, b) a total metals content of less than 10, 5, 2 or 1 ppm, preferably as determined by an ICP spectroscopic method, wherein preferably the degummed feedstock is a refined feedstock.

In one embodiment, there is provided a degummed B. carinata oil, wherein the oil is characterized by at least one of: a) a phosphorus content of less than 10, 5, 2 or 1 ppm, preferably as determined by ³¹P NMR; and, b) a total metals content of less than 10, 5, 2 or 1 ppm, preferably as determined by an ICP spectroscopic method, wherein preferably the degummed oil is a refined oil.

In one embodiment, there is a provided a degummed feedstock, wherein the feedstock is characterized by at least one of: i) a content of calcium of less than 10, 5, 2 or 1 ppm, preferably as determined by an ICP spectroscopic method; ii) a content of iron of less than 2, 1, 0.5 or 0.2 ppm, preferably as determined by an ICP spectroscopic method; and, iii) a content of magnesium of less than 5, 2, 1, 0.5 or 0.2 ppm, preferably as determined by an ICP spectroscopic method, wherein preferably the degummed feedstock is a refined feedstock.

In one embodiment, there is provided a degummed B. carinata oil, wherein the oil is characterized by at least one of: i) a content of calcium of less than 10, 5, 2 or 1 ppm, preferably as determined by an ICP spectroscopic method; ii) a content of iron of less than 2, 1, 0.5 or 0.2 ppm, preferably as determined by an ICP spectroscopic method; and, iii) a content of magnesium of less than 5, 2, 1, 0.5 or 0.2 ppm, preferably as determined by an ICP spectroscopic method, wherein preferably the degummed oil is a refined oil.

In one embodiment, there is provided a degummed feedstock or degummed B. carinata oil as defined above, wherein the feedstock or oil is obtained or obtainable in a process of incubating a feedstock comprising B. carinata oil with a polypeptide having phospholipase A1 activity, as disclosed herein, wherein preferably the degummed feedstock or degummed B. carinata oil is a refined feedstock or refined B. carinata oil.

In one embodiment, a degummed feedstock or degummed B. carinata oil as defined above, is subjected to hydrotreatment, including e.g., at least one of both hydrodeoxygenation and hydroisomerisation, to prepare a hydrotreated vegetable oil (see e.g., WO2016/185046 and WO2016/185047), to be used as biodiesel or aviation biofuel.

In a third aspect, there is a provided a use of a polypeptide having phospholipase A1 activity as defined herein above, whereby the polypeptide having phospholipase A1 activity is used for degumming of a feedstock comprising oil from B. carinata, wherein preferably the feedstock comprises crude B. carinata oil, or wherein, more preferably the feedstock is crude B. carinata oil. In one embodiment, the polypeptide having phospholipase A1 activity is used for degumming in accordance with a process as herein disclosed.

In one embodiment, the polypeptide having phospholipase A1 activity is used in a process for refining a feedstock comprising oil from B. carinata, wherein preferably the feedstock comprises crude B. carinata oil, or wherein, more preferably the feedstock is crude B. carinata oil. In one embodiment, the polypeptide having phospholipase A1 activity is used for refining in accordance with a process as herein disclosed.

In one embodiment, the polypeptide having phospholipase A1 activity is used for reducing the level of phosphorus in a feedstock comprising oil from B. carinata, wherein preferably the feedstock comprises crude B. carinata oil, or wherein, more preferably the feedstock is crude B. carinata oil. In one embodiment, the polypeptide having phospholipase A1 activity is used for reducing the level of phosphorus in accordance with a process as herein disclosed.

In one embodiment, the polypeptide having phospholipase A1 activity is used for reducing the level of metals in a feedstock comprising oil from B. carinata, wherein preferably the feedstock comprises crude B. carinata oil, or wherein, more preferably the feedstock is crude B. carinata oil. In one embodiment, the polypeptide having phospholipase A1 activity is used for reducing the level of metals in accordance with a process as herein disclosed.

The present invention has been illustrated below by non-limiting examples. Modifications and alternative implementations of some parts or elements are possible and are included in the scope of protection as defined in the appended claims.

### EXAMPLES

### Example 1: Composition of Crude Carinata Oil

The composition of crude carinata oil was determined by analyzing the metal content with Inductively Coupled Plasma (ICP), by analyzing Free Fatty Acid (FFA) content with Gas Chromatography (GC), and by analyzing phospholipids profile, being intact phospholipid (PL), lysophospholipid (LPL) and glycerophosphate (GPL), by ³¹P Nuclear Magnetic Resonance (NMR).

### 1.1 Quantitative Determination of Free Fatty Acid (FFA) in Oils

The oil is dissolved in pyridine, which contains pentanoic acid as an internal standard (ISTD). After addition of extra pyridine and N, O-Bis(trimethylsilyl)trifluoroacetamide (BSTFA)/1% trimethylsilyl chloride (TMCS) the sample is heated. This results in the silylation of the hydroxy-groups of the diacylglycerol (DAG), monoacylglycerol (MAG) and the carboxyl group of the free fatty acids (FFA's). The samples are analyzed by Gas Chromatography (GC) with Flame Ionization Detector (GC-FID) using a Programmed Temperature Vaporizing (PTV) injector and equipped with a high temperature column with a DB-5 phase.

### 1.2 Quantitative Determination of Metals in Oils

200-500 mg of sample was weighed accurately into a quartz digestion tube, afterwards a suitable amount of nitric acid (65%, suprapur) is added. The samples are digested using closed microwave digestion using the Milestone UltraWave. The digestion temperature is ramped up in multiple stages up to 250° C. with the internal pressure limited to 110 bar. The digested sample is transferred to a Nunc tube with 2 mL of Hydrochloric acid (30%, suprapur) and approximately 10 mL of MilliQ water. To this 0.5 mL of internal standard solution is added (100 mg NaNO3, 25 mL of 1000 mg/L Sc, Tb, and Au standard solutions. 30 mL of nitric acid (65%, suprapur) diluted to 1000 mL with MilliQ water). The digested sample is diluted further to 50 mL of end volume with MilliQ water.

The data is acquired with the Agilent 7700 ICP-MS equipped with a SPS-4 autosampler, a peltier cooled double pass spray chamber, and micromist nebulizer for sample introduction. Data acquisition parameters: standard plasma mode, He mode collision cell, 1 point peak pattern with 3 replicates per element, and 100 sweeps per replicate, integration time per element 0.20 sec for all elements masses except As (3.00 sec) and Hg (2.00 sec).

The element concentrations in measuring solution are measured with 3-point calibration lines: 0, 125, and 250 μg/L for all elements except As (0, 10, and 20 μg/L), Sb and Tl (0, 25, and 50 μg/L), Hg (0, 2.5, and 5 μg/L), and Pb (0, 5, and 10 μg/L).

Internal standard correction is applied with Sc for element masses up to 97 AMU, and with Tb for the higher element masses.

### 1.3 Quantitative Determination of Phospholipids Using ³¹P NMR

500-1000 mg oil was weighed accurately into a suitable vial, and approximately 10 g cold acetone was added and mixed thoroughly. The oil-acetone mixture was kept at 4° C. for at least 30 min, and then centrifuged for 10 min at 3000 rpm, after which the liquid phase was discarded. The pellet is resuspended in 500 μl buffer (containing 25 g L−1 deoxycholic acid, 5.84 g L−1 EDTA, and 10.9 g L−1 TRIS, buffered using KOH at pH 9.0), and 50 μL of an internal standard solution (containing 10 g L−1 triisopropylphosphate in extraction buffer) was added.

1D ³¹P NMR spectra were recorded on a Bruker Avance III HD spectrometer, operating at a 31p frequency of 161.97 MHz equipped with a nitrogen cooled cryoprobe, at sample temperature of 300K. An inverse gated pulse program (ZGIG) with Waltz16 proton decoupling was used, recording 4 dummy scans, and 128 scans per spectrum, using a 90-degree pulse. An acquisition time of 3.37 s, and a relaxation delay of 11.5 s was used.

The analyte concentrations were calculated relative to triisopropylphosphate. A correction factor was applied to correct for the incomplete relaxation of choline phosphate and ethanolamine phosphate.

### 1.4 Results

The determination results are shown in Table 1.

TABLE 1
Composition of crude carinata oil

| | | | Crude Carinata oil |
|---|---|---|---|
| phosphatidic acid | PA | % | 0.15 |
| phosphatidyl choline | PC | % | 0.05 |
| phosphatidyl ethanolamine | PE | % | 0.05 |
| phosphatidyl inositol | PI | % | 0.03 |
| Total intact phospholipids | Total PL | % | 0.28 |
| Total lyso-phospholipids | Total LPL | % | 0.01 |
| Total glycerol phospholipids | Total GPL | % | — |
| Free fatty acid | FFA | % | 0.74 |
| Phosphorus | P | ppm | 191 |
| Magnesium | Mg | ppm | 34.5 |
| Iron | Fe | ppm | <20 |
| Calcium | Ca | ppm | 137 |

### Example 2: Deep-Degumming/Refining of Crude Carinata Oil Using a Fungal Phospholipase A1

For each individual experiment, 10 g crude carinata oil was weighed into a vial, and heated to 80° C., acid (phosphoric or citric acid) was added to the oil, and incubated for 30 min, after which the oil is allowed to cool down to 60° C. The acid type and dosage are shown in Table 2. Purifine® PLA1 is a phospholipase A1 from Aspergillus niger having the amino acid sequence of SEQ ID NO: 1 (Table 7), which phospholipase can be produced as described in WO2019/215078 or can be obtained from DSM (www.dsm.com/foodspecialties). Purifine® PLA1 is diluted with demi water, and added to the oil, which was immediately followed by high-shear mixing with Ultra Turrax. The enzyme and water dosage are shown in Table 2. After high-shear mixing, the vial was kept at 60° C. for 3 hours, while mixing with a magnetic stirrer at 150 rpm.

TABLE 2
Purifine PLA1 deep-degumming experimental setup.

| | | Citric | | | | |
|---|---|---|---|---|---|---|
| | Phosphoric acid | acid | Temp | Water | Enzyme dosage | Reaction time |
| Exp code | ppm | ppm | ° C. | % | PLAU*/kg oil | hour |
| B-1 | 400 | x | 60 | 3% | 2500** | 3 |
| B-2 | 500 | x | 60 | 3% | 2500 | 3 |
| B-3 | 600 | x | 60 | 3% | 2500 | 3 |
| B-4 | 700 | x | 60 | 3% | 2500 | 3 |
| B-5 | 800 | x | 60 | 3% | 2500 | 3 |
| C-1 | x | 300 | 60 | 3% | 2500 | 3 |
| C-2 | x | 500 | 60 | 3% | 2500 | 3 |

*PLAU = phospholipase A1 units. 1 PLAU is defined as the amount of enzyme that liberates one micromole of free fatty acid per minute under the conditions of the PLA1 assay described in WO 2019/215078.
**2500 PLAU corresponds to 0.6 mg active protein.

After 3 hours incubation, temperature was increased to 85° C. and kept for 30 minutes to inactivate the enzyme. At the end of the inactivation, homogenous samples were taken for the phospholipid content analysis, i.e., phosphatidic acid (PA), phosphatidyl choline (PC), phosphatidyl ethanolamine (PE), and phosphatidyl inositol (PI), and their lyso-form (lyso-phospholipids, LPL), glycerol-form (glycerol phospholipids, GPL), using ³¹P-NMR as described above. Oil samples were taken for FFA analysis using GC method described above. The results are shown in Table 3.

TABLE 3
Purifine PLA1 deep-degumming FFA and phospholipids
composition before centrifugation.

| | FFA | PA | PC | PE | PI | Total PL | Total LPL | Total GPL |
|---|---|---|---|---|---|---|---|---|
| Exp code | % | % | % | % | % | % | % | % |
| crude oil | 0.74 | 0.15 | 0.05 | 0.05 | 0.03 | 0.28 | 0.01 | — |
| B-1 | 1.00 | — | — | — | — | — | 0.05 | 0.03 |
| B-2 | 0.96 | — | — | — | — | — | 0.03 | 0.03 |
| B-3 | 0.95 | — | — | — | — | — | 0.03 | 0.03 |
| B-4 | 0.89 | 0.04 | — | — | — | 0.04 | 0.04 | 0.01 |
| B-5 | 0.88 | 0.05 | — | — | — | 0.05 | 0.05 | — |
| C-1 | 0.88 | 0.03 | — | — | — | 0.03 | 0.02 | 0.07 |
| C-2 | 0.87 | — | — | — | — | — | 0.05 | 0.07 |

After inactivation, the samples were centrifuged in a Sigma 2-15 Howe Centrifuge to separate the oil and gum: the oils were kept at 65-85° C. during the entire process. Centrifugation was for 5 minutes at a max. RCF (Relative centrifugal force) of 2000 G. After centrifugation, oil samples were taken for metal analysis with the ICP method as described above. The results are shown in Table 4.

TABLE 4
Purifine PLA1 deep-degumming oil metal
composition after centrifugation

| | P | Mg | Fe | Ca |
|---|---|---|---|---|
| Sample | ppm | ppm | ppm | ppm |
| Crude oil | 176.0 | 38.2 | 1.8 | 139.0 |
| C-1 | 34.1 | 3.3 | 1.1 | 32.8 |
| C-2 | 9.1 | 1.3 | 0.6 | 6.4 |

Table 4 shows that enzymatic degumming using Purifine PLA1 reduces levels of phosphorus and metals to less than 10 ppm.

The experiment C-2 was repeated at 250 g scale, and oil obtained after centrifugation was washed with 10% hot water by mixing at 85° C. for about 30 min. A separation was done with lab centrifuge at 2000 g for 5 min. The centrifuged oil was then adsorbed using 1% bleaching earth (Tonsil 9192F from Clariant), by mixing the bleaching earth with the centrifuged oil at 105° C. for 30 min. The adsorbed oil was then separated from the bleaching earth by filtration. The resulting oil was analyzed by ³¹P-NMR for phosphorus content, ICP for the metal content and by titration for acid value. The results are shown in Table 5.

TABLE 5
Purifine PLA1 deep-degumming oil C-2, metal composition
after centrifugation and bleaching earth absorption.

| | | | After | After |
|---|---|---|---|---|
| | | | Purifine | bleaching |
| | | Crude | PLA1 | earth |
| Description | | oil | treatment | absorption |
| Calcium, Ca | mg/kg | 151 | 8.3 | 3.7 |
| Iron, Fe | mg/kg | 2.0 | 0.9 | 0.4 |
| Magnesium, Mg | mg/kg | 35.1 | 1.2 | 0.4 |
| Phosphor, P | mg/kg | 188 | 10.6 | <1 |
| Acid value | mg/KOH/g | 3.9 | 4.4 | 4.3 |

### Reference Example 3: Conventional Chemical Deep-Degumming/Refining of Crude Carinata Oil

For each individual experiment, 250 g crude carinata oil was weighed into a conical flask, and heated to 75° C., 500 ppm citric acid was added to the oil, and mixed for 30 min with a magnetic stirrer at 150 rpm. Then 160 ppm sodium hydroxide was added to the oil and mixed at the same condition for 60 min.

Afterwards the samples were centrifuged with Sigma 2-15 Howe Centrifuge to separate the oil and gum: the oils were kept at 65-75° C. during the entire process, the spinning was for 5 minutes at a max. RCF (Relative centrifugal force) of 2000 G. After centrifugation, oil samples were taken for metal analysis with ICP method as described above.

After degumming, the oil was mixed with 1% adsorbent Tonsil 9192F from Clariant for 30 min, and then filtered. The filtered oil were taken for metal analysis with ICP method as described above. The results are shown in Table 6.

TABLE 6
Traditional deep-degumming oil metal composition after handling.

| | P | Mg | Fe | Ca |
|---|---|---|---|---|
| Sample | ppm | ppm | ppm | ppm |
| Crude oil | 176 | 38.2 | 1.8 | 139 |
| After degumming | 112 | 23 | 1.3 | 118 |
| After adsorption | 74 | 14 | 0.9 | 84 |

TABLE 7
Amino acid sequence of the mature A. niger
phospholipase A1.

SEQ ID NO: 1, amino acids 30-298 of Genbank:
XM_001393495
DISSTVLDNIDLFAQYSAAAYCSSNIESTGTTLTCDVGNCPLVEAAGATT
IDEFDDSSSYGDPTGFIAVDPTNELIVLSFRGSSDLSNWIADLDFGLTSV
SSICDGCEMHKGFYEAWEVIADTITSKVEAAVSSYPDYTLVFTGHSYGAA
LAAVAATVLRNAGYTLDLYNFGQPRIGNLALADYITDQNMGSNYRVTHTD
DIVPKLPPELLGYHHFSPEYWITSGNDVTVTTSDVTEVVGVDSTAGNDGT
LLDSTTAHRWYTIYISECS

1. A process for reducing an amount of at least one of intact phospholipids and metals in a feedstock comprising oil from Brassica carinata, the process comprising incubating the feedstock with a polypeptide having phospholipase A1 activity, wherein the polypeptide comprises an amino acid sequence having at least 50%, optionally at least 75%, identity to SEQ ID NO: 1.

2. The process according to claim 1, wherein the polypeptide reduces at least 85% of the amount of intact phospholipids originally present in a crude B. carinata oil when the polypeptide is incubated with the crude B. carinata oil in an amount of 2500 PLAU/kg oil in the presence of 300-500 ppm citric acid, at a temperature of 60° C. for 3 hours.

3. The process according to claim 1, wherein the polypeptide is an Aspergillus phospholipase A1, optionally an A. niger phospholipase A1, or optionally the polypeptide comprises the amino acid sequence of SEQ ID NO:1.

4. The process according to claim 1, wherein the feedstock is incubated with the polypeptide having phospholipase A1 activity in the presence of an acid, wherein, optionally the acid is an acid selected from the group consisting citric acid, phosphoric acid, acetic acid, tartaric acid, succinic acid, and mixtures thereof.

5. The process according to claim 4, wherein the acid is present in an amount of 100-1000 ppm, optionally 200-800 ppm, optionally 400-600 ppm, or optionally 400-600 ppm citric acid.

6. The process according to claim 1, wherein the feedstock is incubated with the polypeptide having phospholipase A1 activity at a temperature in the range of 50-70° C., optionally at temperature in the range of 55-65° C.

7. The process according to claim 1, further comprising separating the phosphorous-containing components from the processed feedstock, optionally by centrifugation.

8. The process according to claim 1, further comprising combining the processed feedstock with an adsorption medium to generate a slurry and separating the adsorbed processed feedstock from the adsorption medium.

9. The process according to claim 8, wherein the adsorption medium is a bleaching earth.

10. The process according to claim 1, wherein the process is a process for degumming and/or refining the feedstock.

11. The process according to claim 1, wherein the feedstock comprises crude B. carinata oil, or wherein optionally the feedstock is crude B. carinata oil.

12. A degummed feedstock comprising B. carinata oil, wherein the feedstock is characterized by at least one of:
a) a phosphorus content of less than 10, 5, 2 or 1 ppm, optionally as determined by ³¹P NMR; and,
b) a total metals content of less than 10, 5, 2 or 1 ppm, optionally as determined by an ICP spectroscopic method,
and wherein optionally the degummed feedstock is degummed B. carinata oil.

13. The degummed feedstock or degummed B. carinata oil according to claim 12, wherein the feedstock or the oil is characterized by at least one of:
i) a content of calcium of less than 10, 5, 2 or 1 ppm, optionally as determined by an ICP spectroscopic method;
ii) a content of iron of less than 2, 1, 0.5 or 0.2 ppm, optionally as determined by an ICP spectroscopic method; and,
iii) a content of magnesium of less than 5, 2, 1, 0.5 or 0.2 ppm, optionally as determined by an ICP spectroscopic method.

14. The degummed feedstock or degummed B. carinata oil according to claim 12, wherein the degummed feedstock or degummed oil is obtainable in a process for reducing an amount of at least one of intact phospholipids and metals in a feedstock comprising oil from Brassica carinata, the process comprising incubating the feedstock with a polypeptide having phospholipase A1 activity, wherein the polypeptide comprises an amino acid sequence having at least 50%, optionally at least 75%, identity to SEQ ID NO: 1.

15. A method of:
a) degumming;
b) refining;
c) reducing the level of phosphorus in; or;
d) reducing the level of metals in
a feedstock comprising oil from B. carinata, wherein optionally the feedstock comprises crude B. carinata oil, or wherein optionally the feedstock is crude B. carinata oil,
the method comprising incubating the feedstock with a polypeptide having phospholipase A1 activity.


SEQUENCE LISTING (1 sequence)

SEQ ID NO: 1 - protein, 269 aa, Aspergillus niger
DISSTVLDNIDLFAQYSAAAYCSSNIESTGTTLTCDVGNCPLVEAAGATTIDEFDDSSSYGDPTGFIAVDPTNELIVLSFRGSSDLSNWIADLDFGLTSVSSICDGCEMHKGFYEAWEVIADTITSKVEAAVSSYPDYTLVFTGHSYGAALAAVAATVLRNAGYTLDLYNFGQPRIGNLALADYITDQNMGSNYRVTHTDDIVPKLPPELLGYHHFSPEYWITSGNDVTVTTSDVTEVVGVDSTAGNDGTLLDSTTAHRWYTIYISECS